(19) United States
(12) Patent Application Publication
(10) Pub. No.: US 2025/0134369 A1
(43) Pub. Date: May 1, 2025

(54) HUMAN-EYE STATE DETECTION DEVICE AND HUMAN-EYE STATE DETECTION METHOD THEREOF

(71) Applicant: Wistron Corp., New Taipei City (TW)

(72) Inventors: Chin Hao HSU, New Taipei City (TW); Chih Hao CHIU, New Taipei City (TW); Chien Hua CHEN, New Taipei City (TW); Hsiu Mei LIN, New Taipei City (TW)

(21) Appl. No.: 18/435,041

(22) Filed: Feb. 7, 2024

(30) Foreign Application Priority Data
Oct. 31, 2023 (TW) 112141663

(51) Int. Cl. A61B 3/113; A61B 3/14; G06T 7/00; G16H 50/20

(52) U.S. Cl. CPC A61B 3/113; A61B 3/14; G06T 7/0012; G16H 50/20; G06T 2207/30201

(57) ABSTRACT

A human-eye state detection device is provided, which includes an image capturing device and a processor. The image capturing device is configured to continuously capture a plurality of frames of facial images of a user. The processor determines an eye area from the frames of facial images, and calculates an eye-opening average based on the eye area. The processor repeatedly updates the eye-opening threshold based on the average eye-opening value. In response to the average eye-opening value being less than the eye-opening threshold, the processor determines that the user is in an eye-closing state.

### CROSS REFERENCE TO RELATED APPLICATIONS

This application claims priority of Taiwan Patent Application No. 112141663, filed on Oct. 31, 2023, the entirety of which is incorporated by reference herein.

### BACKGROUND OF THE INVENTION

### Field of the Invention

The present disclosure is related to a human-eye state detection device and a human-eye state detection method thereof, and more particularly it is related to a human-eye state detection device and a human-eye state detection method thereof that continuously updates the eye-opening threshold.

### Description of the Related Art

Methods designed to recognize the open and closed state of the human eye are important in a number of fields, including automotive safety and medicine. In driver monitoring systems, methods to detect human-eye state are often needed to monitor the driver's eyes, and to determine whether the driver is in a state of physiological fatigue based on the movements of the eyelids, whereupon if the driver appears to be fatigued, the system can issue a warning, thereby contributing to road safety.

However, there are many technical problems during the process of detecting the human-eye state using these detection methods. Because eye size varies with different people (as well as due to different angles, different distances, and other environmental factors, in addition to interference from external factors such as whether eyes glasses are being worn, changes in light and shade in the picture, etc.), traditional human-eye state detection methods are not able to consistently determine eye state correctly. Therefore, there is a need for a human-eye state detection device and a human-eye state detection method that can accurately determine the state of human eyes under any circumstances, so as to solve the above problems.

### BRIEF SUMMARY OF THE INVENTION

A human-eye state detection device and a human-eye state detection method thereof are proposed herein. By continuously updating the eye-opening threshold values of the left eye and the right eye, there is a representative eye-opening threshold value as the basis for determination in the process of detecting the state of the human eyes. In addition, by dynamically adjusting the eye-opening thresholds of the left and right eyes, it helps to significantly reduce the impact of the external environment. Regardless of whether the user's left and right eyes are the same size, whether the eyes are disturbed by the shadow of other things (such as hair), whether the user wears glasses, whether the lens has an elevation or depression angle, and other individual factors and environmental factors, it can accurately determine the state of human eyes. Furthermore, the possibility of misjudgment would be significantly reduced since the state of eyes of the user is determined based on the states of the left eye and the right eye, thereby improving the user experience.

In an embodiment, a human-eye state detection device is provided. The human-eye state detection device comprises an image capturing device and a processor. The image capturing device continuously captures a plurality of frames of facial images of a user. The processor determines an eye area from the frames of facial images and calculating the average eye-opening value based on the eye area. The processor repeatedly updates the eye-opening threshold value based on the average eye-opening value. In response to the average eye-opening value being less than the eye-opening threshold value, the processor determines that the user is in an eye-closing state.

According to an embodiment of the present disclosure, in response to the processor determining that an execution condition has been met, the processor calculates an eye-opening value from the eye area of each frame and generates the average eye-opening value by averaging the eye-opening values of a specific amount of frames at a first point of time. The average eye-opening value generated at the first point of time is a first average eye-opening value. The processor multiplies the first average eye-opening value by a ratio to generate an initial eye-opening threshold value and takes the initial eye-opening threshold value as the eye-opening threshold value.

According to an embodiment of the present disclosure, after the processor takes the initial eye-opening threshold value as the eye-opening threshold value, the processor uses the average of the eye-opening values of the specific amount of frames at a second point of time to generate a real-time average eye-opening value, thereby updating the average eye-opening value to a second average eye-opening value. The eye-opening threshold value is the second average eye-opening value multiplied by the ratio. The second average eye-opening value is the average of the first average eye-opening value and the real-time average eye-opening value.

According to an embodiment of the present disclosure, the processor further averages a plurality of eye-closing values within a predetermined period to generate an average eye-closing value. The eye-closing value is the eye-opening value being less than the eye-opening threshold value within a predetermined period. The processor updates the eye-opening threshold value to a weighted average of the average eye-opening value and the average eye-closing value.

According to an embodiment of the present disclosure, the eye-opening threshold value is the sum of the average eye-opening value multiplied by a first weighted factor and the average eye-closing value multiplied by a second weighted factor. The sum of the first weighted factor and the second weighted factor is 1. In response to the processor operating in a first sensitivity mode, the first weighted factor is a first value. In response to the processor operating in a second sensitivity mode, the first weighted factor is a second value. The first value and the second value are different. In response to the processor operating in the first sensitivity mode, the ratio is a first ratio value. In response to the processor operating in the second sensitivity mode, the ratio is a second ratio value. The first ratio value and the second ratio value are different.

According to an embodiment of the present disclosure, the processor further determines whether the second average eye-opening value is less than the initial eye-opening threshold value. In response to the second average eye-opening value being less than the initial eye-opening threshold value, the processor adjusts the average eye-opening value to the average of the first average eye-opening value and the second average eye-opening value.

According to an embodiment of the present disclosure, in response to the second average eye-opening value not being less than the initial eye-opening value, the processor further determines whether the eye-opening threshold value exceeds the first average eye-opening value. In response to the eye-opening threshold exceeding the first average eye-opening value, the processor adjusts the average eye-opening value to the average of the first average eye-opening value and the second average eye-opening value. In response to the eye-opening threshold not exceeding the first average eye-opening value, the processor repeatedly updates the average eye-opening value in real-time. The eye-opening threshold value varies with the average eye-opening value.

According to an embodiment of the present disclosure, the human-eye state detection device further comprises a detection device and a memory. The detection device detects a moving speed. A memory stores the average eye-opening value, the first average eye-opening value, the initial eye-opening value, and the eye-opening threshold value. In response to the moving speed exceeding a predetermined speed, the processor determines that the execution condition has been met. In response to the execution condition not being met, the processor erases the average eye-opening value, the first average eye-opening value, the initial eye-opening threshold value, and the eye-opening threshold value in the memory.

According to an embodiment of the present disclosure, the processor further determines, by morphology, whether the eye area is in an eye-opening state. In response to the eye area is in the eye-opening state and the moving speed exceeding the predetermined speed, the processor determines that the execution condition has been met.

According to an embodiment of the present disclosure, the processor further determines a left eye area and a right eye area from the eye area and generates the average eye-opening values and the eye-opening threshold values corresponding to the left eye area and the right eye area based on the left eye area and the right eye area respectively. In response to the processor determining that the average eye-opening value of the left eye area and the average eye-opening value of the right eye area are both less than the corresponding eye-opening threshold values, the processor determines that the user is in the eye-closing state.

In another embodiment, a human-eye state detection method adapted to a human-eye state detection device is provided. The human-eye state detection device comprises an image capturing device. The human-eye state detection method comprises the following steps. A plurality of frames of facial images of a user are continuously captured using the image capturing device. The eye area can be determined from the frames of the facial images. The average eye-opening value is calculated based on the eye area. Based on the average eye-opening value, the eye-opening threshold value is updated in real time. In response to the average eye-opening value being less than the eye-opening threshold value, it is determined that the user is in an eye-closing state.

According to an embodiment of the present disclosure, the human-eye state detection method further comprises the following steps. It is determined whether an execution condition is met. In response to determining that the execution condition has been met, an eye-opening value from the eye area of each frame is calculated and the eye-opening values of a specific amount of frames are averaged at a first point of time to generate the average eye-opening value. The average eye-opening value generated at the first point of time is a first average eye-opening value. The first average eye-opening value is multiplied by a ratio to generate an initial eye-opening threshold value as the eye-opening threshold value.

According to an embodiment of the present disclosure, the human-eye state detection method further comprises the following steps.

After the step of multiplying the first average eye-opening value by the ratio to generate the initial eye-opening threshold value as the eye-opening threshold value, a real-time average eye-opening value is generated by averaging the eye-opening values of the specific amount of frames at a second point of time to update the average eye-opening value to a second average eye-opening value. The eye-opening threshold value is the second average eye-opening value multiplied by the ratio. The second average eye-opening value is the average of the first average eye-opening value and the real-time average eye-opening value.

According to an embodiment of the present disclosure, the human-eye state detection method further comprises the following steps. The number of eye-closing values within a predetermined period are averaged in order to generate the average eye-closing value. The eye-opening value within the predetermined period being less than the eye-opening threshold value is the eye-closing value. The eye-opening threshold value is updated to a weighted average of the average eye-opening value and the average eye-closing value.

According to an embodiment of the present disclosure, the human-eye state detection method further comprises the following steps. The eye-opening threshold value is the sum of the average eye-opening value multiplied by a first weighted factor and the average eye-closing value multiplied by a second weighted factor. The sum of the first weighted factor and the second weighted factor is 1. In response to the human-eye state detection device operating in a first sensitivity, the first weighted factor is a first value. In response to the human-eye state detection device operating in a second sensitivity, the second weighted factor is a second value. The first value and the second value are different. In response to the processor operating in the first sensitivity, the ratio is a first ratio value. In response to the processor operating in the second sensitivity, the ratio is a second ratio value. The first ratio value and the second ratio value are different.

According to an embodiment of the present disclosure, the human-eye state detection method further comprises the following steps. It is determined whether the second average eye-opening value is less than the initial eye-opening threshold value. In response to the second average eye-opening value being less than the initial eye-opening threshold value, the average eye-opening value is adjusted to the average of the first average eye-opening value and the second average eye-opening value.

According to an embodiment of the present disclosure, the human-eye state detection method further comprises the following steps. In response to the second average eye-opening value not being less than the initial eye-opening threshold value, it is further determined whether the eye-opening threshold value exceeds the first average eye-opening value. In response to the eye-opening threshold value exceeding the first average eye-opening value, the average eye-opening value is adjusted to the average of the first average eye-opening value and the second average eye-opening value. In response to the eye-opening threshold value not exceeding the first average eye-opening value, the average eye-opening value is repeatedly updated. The eye-opening threshold value varies with the average eye-opening value.

According to an embodiment of the present disclosure, the human-eye state detection method further comprises the following steps. A moving speed is detected using a detection device. The average eye-opening value, the first average eye-opening value, the initial eye-opening threshold value, and the eye-opening threshold value are stored by using a memory. In response to the moving speed exceeding a predetermined speed, it is determined that the execution condition has been met. In response to the execution condition not being satisfied, the average eye-opening value, the first average eye-opening value, the initial eye-opening threshold value, and the eye-opening threshold value that are stored are erased.

According to an embodiment of the present disclosure, the human-eye state detection method further comprises the following steps. It is determined whether the eye area is in an eye-opening state by morphology. In response to the eye area being in the eye-opening state and the moving speed exceeding the predetermined speed, determining that the execution condition has been met.

According to an embodiment of the present disclosure, the human-eye state detection method further comprises the following steps. It is determined a left eye area and a right eye area from the eye area. The average eye-opening values and the eye-opening threshold values corresponding to the left eye area and the right eye area are generated based on the left eye area and the right eye area. In response to determining that the average eye-opening value corresponding to the left eye area and the average eye-opening value corresponding to the right eye area both are less than the corresponding eye-opening threshold values, it is determining that the user is in the eye-closing state.

A detailed description is given in the following embodiments with reference to the accompanying drawings.

### BRIEF DESCRIPTION OF DRAWINGS

The invention can be more fully understood by reading the subsequent detailed description and examples with references made to the accompanying drawings, wherein:

FIG. 1 is a block diagram showing a human-eye state detection device in accordance with an embodiment of the present disclosure;

FIG. 2 is a schematic diagram showing an image signal in accordance with an embodiment of the present disclosure;

FIG. 3A is a flow chart showing the analysis process of an image signal in accordance with an embodiment of the present disclosure;

FIG. 3B is a schematic diagram showing the analysis process of an image signal in accordance with an embodiment of the present disclosure;

FIG. 4 is a flow chart showing a human-eye state detection method in accordance with an embodiment of the present disclosure;

FIG. 5 is a flow chart showing an update procedure in accordance with an embodiment of the present disclosure;

FIG. 6 is a schematic diagram showing an eye-opening value in accordance with an embodiment of the present disclosure;

FIG. 7 is a schematic diagram showing the average eye-opening value and the eye-opening threshold value in accordance with an embodiment of the present disclosure;

FIG. 8 is a schematic diagram showing the average eye-opening value and the eye-opening threshold value in accordance with another embodiment of the present disclosure; and

FIG. 9 is a flowchart showing a determination procedure in accordance with′ an embodiment of the present disclosure.

### DETAILED DESCRIPTION OF THE INVENTION

The following description is made for the purpose of illustrating the general principles of the disclosure and should not be taken in a limiting sense. The scope of the disclosure is determined by reference to the appended claims.

In the following detailed description, for purposes of explanation, numerous specific details and embodiments are set forth in order to provide a thorough understanding of the present disclosure. The use of like and/or corresponding numerals in the drawings of different embodiments does not suggest any correlation between different embodiments.

In addition, in some embodiments of the present disclosure, terms concerning attachments, coupling and the like, such as “connected” and “interconnected,” refer to a relationship wherein structures are secured or attached to one another either directly or indirectly (for example, electrically connection) via intervening structures, as well as both movable or rigid attachments or relationships, unless expressly described otherwise.

In addition, in this specification, relative spatial expressions are used. For example, “lower”, “bottom”, “higher” or “top” are used to describe the position of one element relative to another. It should be appreciated that if a device is flipped upside down, an element that is “lower” will become an element that is “higher”.

It should be understood that, although the terms first, second, third etc. may be used herein to describe various elements, components, regions, layers, portions and/or sections, these elements, components, regions, layers, portions and/or sections should not be limited by these terms. These terms are only used to distinguish one element, component, region, layer, portion or section from another element, component, region, layer or section. Thus, a first element, component, region, layer, portion or section in the specification could be termed a second element, component, region, layer, portion or section in the claims without departing from the teachings of the present disclosure.

It should be understood that this description of the exemplary embodiments is intended to be read in connection with the accompanying drawings, which are to be considered part of the entire written description. The drawings are not drawn to scale. In addition, structures and devices are shown schematically in order to simplify the drawing.

The terms “approximately”, “about” and “substantially” typically mean a value is within a range of +/−20% of the stated value, more typically a range of +/−10%, +/−5%, +/−3%, +/−2%, +/−1% or +/−0.5% of the stated value. The stated value of the present disclosure is an approximate value. Even there is no specific description, the stated value still includes the meaning of “approximately”, “about” or “substantially”.

Unless defined otherwise, all technical and scientific terms used herein have the same meaning as commonly understood by one of ordinary skill in the art to which this disclosure belongs. It should be appreciated that, in each case, the term, which is defined in a commonly used dictionary, should be interpreted as having a meaning that conforms to the relative skills of the present disclosure and the background or the context of the present disclosure, and should not be interpreted in an idealized or overly formal manner unless so defined.

In addition, in some embodiments of the present disclosure, terms concerning attachments, coupling and the like, such as “connected” and “interconnected,” refer to a relationship wherein structures are secured or attached to one another either directly or indirectly (for example, electrically connection) via intervening structures, as well as both movable or rigid attachments or relationships, unless expressly described otherwise.

In the drawings, similar elements and/or features may have the same reference number. Various components of the same type can be distinguished by adding letters or numbers after the component symbol to distinguish similar components and/or similar features.

In addition, in some embodiments of the present disclosure, terms concerning attachments, coupling and the like, such as “connected” and “interconnected,” refer to a relationship wherein structures are secured or attached to one another either directly or indirectly (for example, electrically connection) via intervening structures, as well as both movable or rigid attachments or relationships, unless expressly described otherwise.

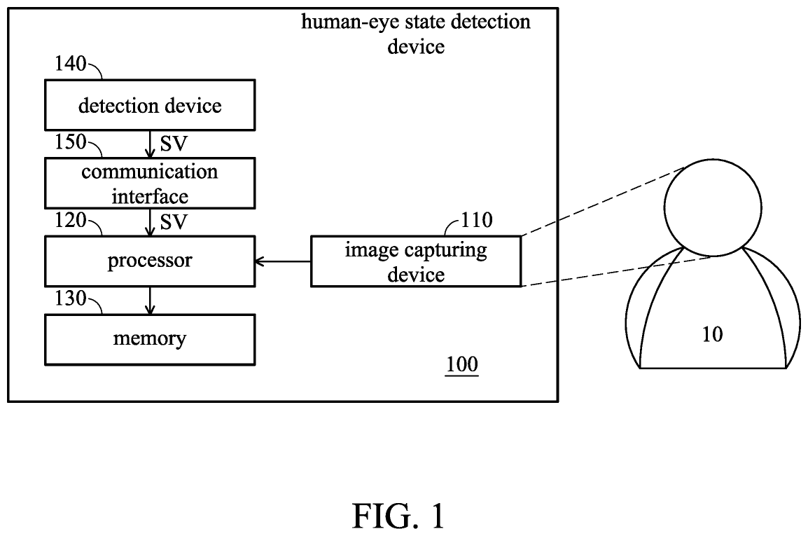

FIG. 1 is a block diagram showing a human-eye state detection device in accordance with an embodiment of the present disclosure. As shown in FIG. 1, the human-eye state detection device 100 includes an image capturing device 110, a processor 120, and a memory 130. The image capturing device 110 is configured to continuously capture multiple frames of facial images of the user 10 to generate image signals. According to some embodiments of the present disclosure, the image capturing device 110 may be a camera with a photosensitive coupling device, such as Charge-coupled Device (CCD) or a complementary metal-oxide semiconductor (CMOS) photosensitive device.

The processor 120 receives the image signals generated by the image capturing device 110, analyzes the image signal, and then determines the state of the user's 10 eyes. The detailed determination procedure of the human-eye state will be thoroughly described in the following paragraphs. According to some embodiments of the present disclosure, the processor 120 may be implemented as an Application Specific Integrated Circuit (ASIC), a Field Programmable Gate Array (FPGA), or other logic circuits, but the present disclosure is not limited thereto.

The memory 130 is configured to store the image signals received by the processor 120, and to store the data generated by the processor 120 in the determination process of the state of the user's 10 eyes. According to some embodiments of the present disclosure, the memory 130 may be a Static Random-Access Memory (SRAM), a Dynamic Random-Access Memory (DRAM), or a flash memory., Electronically-Erasable Programmable Read-Only Memory (EEPROM) or any other machine-readable memory.

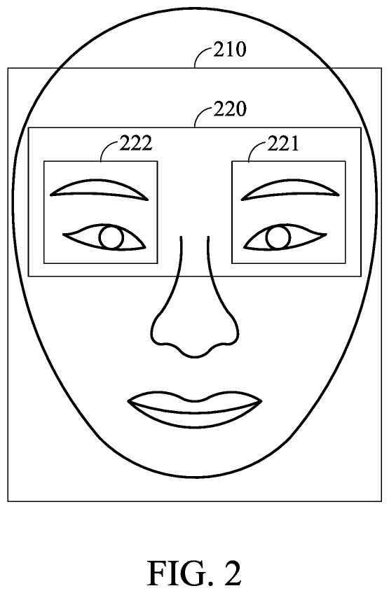

FIG. 2 is a schematic diagram showing an image signal in accordance with an embodiment of the present disclosure. In one embodiment of the present disclosure, when the processor 120 receives the image signals generated by the image capturing device 110, the processor 120 analyzes the image signals to determine the user's face area 210 and to determine the eye area 220 based on the face area 210, where the eye area 220 is the region of interest (ROI).

It should be noted that the eye area 220 refers to the human eye and the area around it, which may include areas such as frames, eyebrows, eyes, and eyelids. According to some embodiments of the present disclosure, the eye area 220 may be further divided into a left eye area 221 and a right eye area 222, where the left eye area 221 and the right eye area 222 are the region of interest.

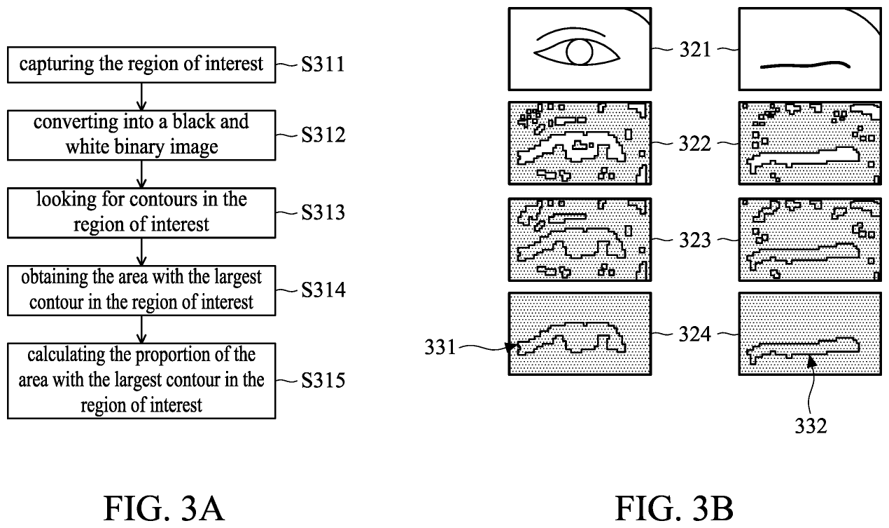

FIG. 3A is a flow chart showing the analysis process of an image signal in accordance with an embodiment of the present disclosure. FIG. 3B is a schematic diagram showing the analysis process of an image signal in accordance with an embodiment of the present disclosure. In step S311, the processor 120 captures the eye area 220 of the face area 210 in FIG. 2 as the region of interest. As shown in the embodiment of the first schematic diagram 321 in FIG. 3B, the processor 120 captures the right eye area 222 in FIG. 2 as the region of interest, where the eye in the right eye area 222 may be in an eye-opening state or an eye-closing state. In order to simplify the explanation, the right eye area 222 is illustrated for simplicity of explanation, and is not limited thereto in any form.

In step S312, the processor 120 converts the region of interest into a black-and-white binary image. As shown in the second schematic diagram 322 of FIG. 3B, the processor 120 converts the image of the first schematic diagram 321 into a black-and-white binary image. In step S313, the processor 120 looks for contours in the region of interest. As shown in the third schematic diagram 323 of FIG. 3B, the processor 120 depicts all the contours in the second schematic diagram 322. In other words, processor 120 looks for all contours in right eye area 222.

Next, in step S314, the processor 120 obtains the area with the largest contour in the region of interest. As shown in the fourth schematic diagram 324 of FIG. 3B, the processor 120 obtains the area of the first maximum contour 331 or the second maximum contour 332, where the first maximum contour 331 and the second maximum contour 332 correspond to the right eye area 222 in the eye-opening state and the eye-closing state respectively.

Finally, in step S315, the processor 120 calculates the ratio of the area of the maximum contour in the region of interest. In the embodiment of FIG. 3B, the processor 120 calculates the ratio of the area of the first largest contour 331 to the right eye area 222, or the ratio of the second largest contour 332 to the right eye area 222, where the ratio is hereinafter referred to as is the eye-opening value, and the ratio is less than 1.

Referring to FIG. 1, the human-eye state detection device 100 further includes a detection device 140 and a communication interface 150. The detection device 140 is configured to detect the moving speed of the human-eye state detection device 100 to generate a speed signal SV, and provide the speed signal SV to the processor 120 through the communication interface 150. According to an embodiment of the present disclosure, the processor 120 determines whether the execution condition is met according to the moving speed of the speed signal SV to determine whether to control the image capturing device 110 to start capturing the facial image of the user 10.

According to one embodiment of the present disclosure, the human-eye state detection device 100 is installed on a mobile vehicle, and the detection device 140 is configured to detect the moving speed of the mobile vehicle. According to some embodiments of the present disclosure, the communication interface 150 may be a Controller Area Network (CAN), or other communication interface configured for data transmission.

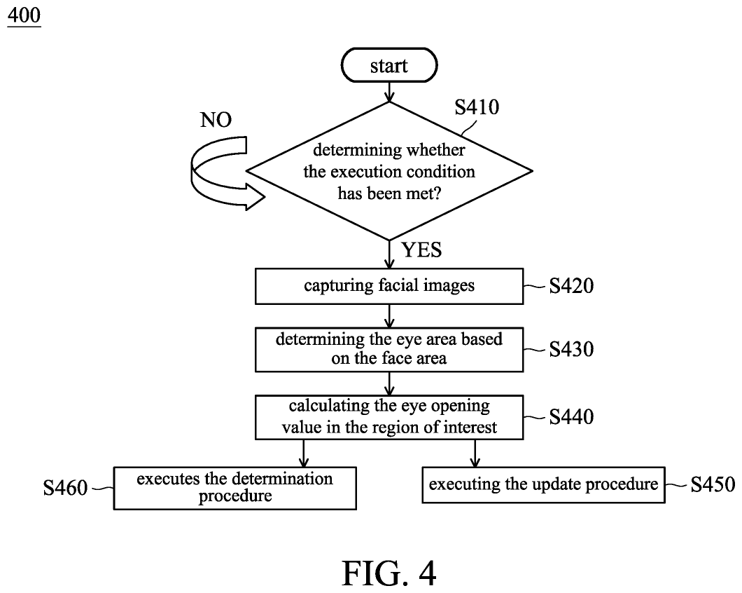

FIG. 4 is a flow chart showing a human-eye state detection method in accordance with an embodiment of the present disclosure. The following is a description of the human-eye state detection method 400 in FIG. 4, and will be combined with FIG. 1 and FIG. 2 for detailed explanation.

In step S410, the processor 120 in FIG. 1 determines whether the execution condition has been met. According to an embodiment of the present disclosure, the processor 120 determines whether the moving speed represented by the speed signal SV exceeds a predetermined speed based on the speed signal SV generated by the detection device 140, and then determines whether to continue to execute the subsequent steps of the human-eye state detection method 400. When the moving speed does not exceed the predetermined speed, the processor 120 does not execute subsequent steps of the human-eye state detection method 400 and repeatedly determines whether the execution condition has been met.

According to some embodiments of the present disclosure, the predetermined speed may be set to be less than 15 kilometers per hour, but the present disclosure is not limited thereto. According to other embodiments of the present disclosure, the predetermined speed may be approximately 10 kilometers per hour.

According to another embodiment of the present disclosure, the processor 120 may first control the image capturing device 110 to capture the face area 210 of the user 10, and determines whether the eye area 220 of the user 10 is open by morphology. When the eye area 220 is in the eye-opening state, the processor 120 determines that the execution condition has been met, and then executes the subsequent steps of the human-eye state detection method 400. When the eye area 220 is in the eye-closing state, the processor 120 determines that the execution condition has not been met and repeatedly determines whether the eye area 220 of the user 10 is in the eye-opening state.

According to other embodiments of the present disclosure, the processor 120 can determine whether to execute the subsequent steps of the human-eye state detection method 400 based on whether the moving speed exceeds a predetermined speed and whether the user 10 is in an eye-opening state by morphology. When the moving speed exceeds the predetermined speed and the user 10 is determined to be in an eye-opening state by morphology, the processor 120 determines that the execution condition has been met and starts executing the subsequent steps of the human-eye state detection method 400. Otherwise, the processor 120 repeatedly executes step S410.

When it is determined in step S410 that the execution condition has been satisfied, the processor 120 controls the image capturing device 110 to continuously capture multiple frames of facial images of the user 10 (step S420), where the facial images include the face area 210 in FIG. 2. Next, the processor 120 determines the eye area 220 based on the face area 210 (step S430), where the processor 120 uses the eye area 220 as a region of interest (ROI).

According to some embodiments of the present disclosure, the processor 120 may further determine the left eye area 221 and the right eye area 222 from the eye area 220, and takes the left eye area 221 and the right eye area 222 as the regions of interest. When the processor 120 takes both the left eye area 221 and the right eye area 222 as regions of interest, the processor 120 may execute the human-eye state detection method 400 for the left eye area 221 and the right eye area 222 respectively.

Subsequently, the processor 120 calculates the eye-opening value in the region of interest (step S440). As shown in FIG. 3A, the processor 120 calculates the ratio of the maximum contour area to the area of the region of interest in the left eye area 221 and of the right eye area 222 respectively, and serves as the eye-opening value of the left eye and the eye-opening value of the right eye respectively. The detailed process is shown in FIGS. 3A-3B which will not be repeated herein.

After step S440, the processor 120 simultaneously executes the update procedure (step S450) and the determination procedure (step S460). According to some embodiments of the present disclosure, when executing step S420 to step S460, the processor 120 simultaneously continues to determine whether the execution condition is met. When it is determined that the execution condition has not been met, the processor 120 may interrupt the human-eye state detection method 400 at any time and return to step S410 to repeatedly determine whether the execution condition has been met again.

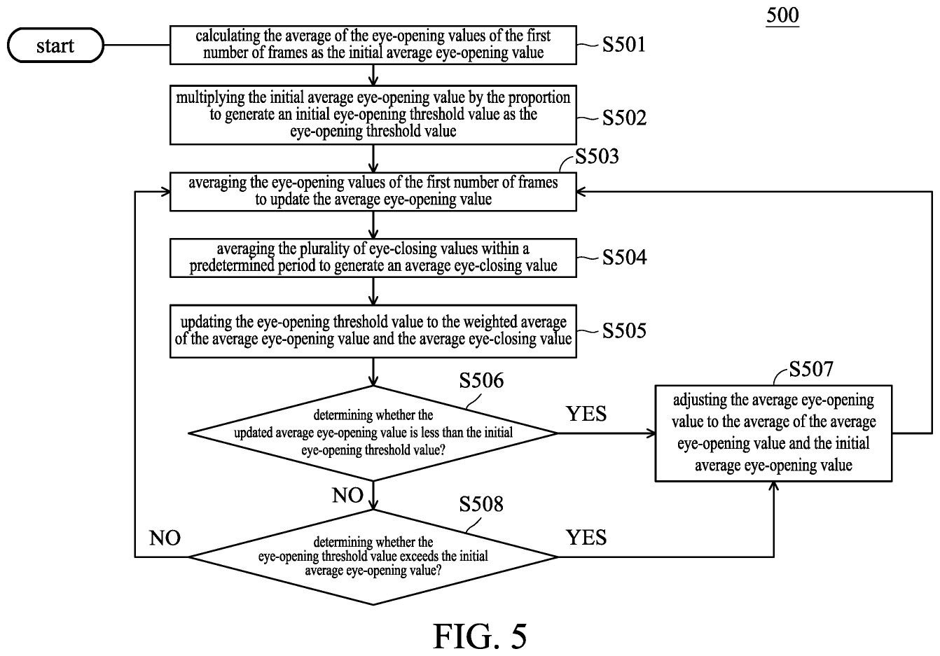

FIG. 5 is a flow chart showing an update procedure in accordance with an embodiment of the present disclosure. According to some embodiments of the present disclosure, the update procedure 500 in FIG. 5 is configured to update the eye-opening threshold values of the left eye area 221 and the right eye area 222. In other words, the left eye area 221 and the right eye area 222 have different eye-opening threshold values. In order to facilitate the explanation below, the update procedure 500 will be illustrated with the right eye area 222 as an example for explanation, but not intended to be limited thereto in any form.

First, the processor 120 calculates the average of the eye-opening values of the first number of frames to obtain the initial average eye-opening value (step S501). According to some embodiments of the present disclosure, the first number may be 10 to 20. In other words, when the human-eye state detection method 400 first captures the facial image of the user 10, the processor 120 averages the eye-opening values of the right eye area 222 in 10 to 20 frames to generate an initial average eye-opening value.

Next, the processor 120 multiplies the initial average eye-opening value by a ratio to generate an initial eye-opening threshold value (step S502), and takes the initial eye-opening threshold value as the eye-opening threshold value. According to an embodiment of the present disclosure, when the processor 120 determines that the average eye-opening value is less than the eye-opening threshold value at any time, the processor 120 determines that the user 10 is in the eye-closing state. In other words, the eye-opening threshold is a standard configured to determine whether the user 10 is in an eye-opening state or an eye-closing state.

According to some embodiments of the present disclosure, when the processor 120 operates in the low sensitivity mode, the initial average eye-opening value is multiplied by the first ratio to generate an initial eye-opening threshold value. When the processor 120 operates in the medium sensitivity mode, the initial average eye-opening value is multiplied by the second ratio to generate an initial eye-opening threshold value. When the processor 120 operates in the high sensitivity mode, the initial average eye-opening value is multiplied by the third ratio to generate an initial eye-opening threshold value. The third ratio exceeds the second ratio, the second ratio exceeds the first ratio, and the first ratio, the second ratio and the third ratio do not exceed 1.

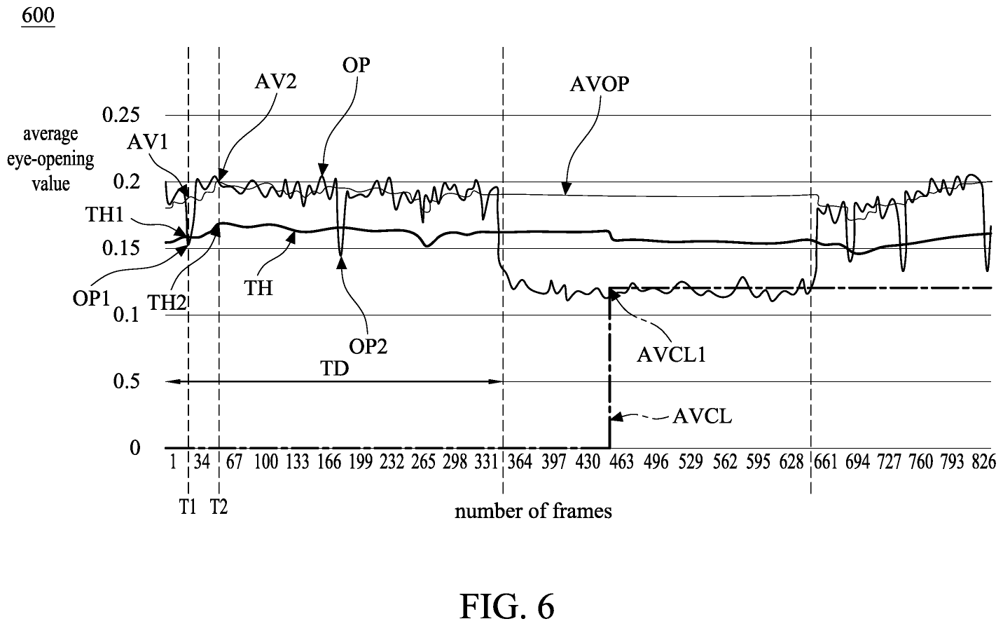

After the initial eye-opening threshold value is generated (i.e., step S502), the processor 120 continues to calculate the average of the eye-opening values of the first number of frames to update the average eye-opening value (step S503). The detailed operations from step S501 to step S503 will be explained in detail with reference to FIG. 6.

FIG. 6 is a schematic diagram showing an eye-opening value in accordance with an embodiment of the present disclosure. As shown in FIG. 6, the vertical axis of the waveform 600 is the eye-opening value of the region of interest, and the horizontal axis is the number of frames. As the image capturing device 110 continuously captures multiple frames of facial images, the processor 120 calculates the eye-opening value OP corresponding to different frames, and generates the average eye-opening value AVOP and the average eye-closing value AVCL based on the eye-opening value OP.

As shown in FIG. 6, the processor 120 averages the eye-opening values OP of the first number of frames at the first point of time T1 to generate a first average eye-opening value AV1, averages the eye-opening values OP of the first number of frames at the second point of time T2 to generate a real-time average eye-opening value AVR, and updates the second average eye-opening value AV2 using the first average eye-opening value AV1 and the real-time average eye-opening value AVR.

According to an embodiment of the present disclosure, when the processor 120 generates the initial average eye-opening value at the first point of time T1, the processor 120 averages the eye-opening values of the first number of frames before the first point of time T1 to generate the first average eye-opening value AV1, where the first average eye-opening value AV1 is the initial average eye-opening value. Furthermore, the processor 120 further multiplies the first average eye-opening value AV1 by a ratio to generate a first eye-opening threshold value TH1, where the first eye-opening threshold value TH1 is the initial eye-opening threshold value.

According to another embodiment of the present disclosure, the processor 120 calculates the second average eye-opening value AV2 again at the second point of time T2 after the first point of time T1. The average of the eye-opening values OP of the first number of frames at the second point of time T2 is the real-time average eye-opening value AVR, and the second average eye-opening value AV2 is the average of the real-time average eye-opening value AVR and the first average eye-opening value AV1, which is shown in Eq. 1:

$$AV 2 = \frac{A V R + AV 1}{2} \qquad ( Eq . l )$$

In other words, when the first average eye-opening value AV1 is the initial average eye-opening value, the second average eye-opening value AV2 is the average of the initial average eye-opening value and the real-time average eye-opening value AVR. When the first average eye-opening value AV1 is not the initial average eye-opening value, the second average eye-opening value AV2 is still the average of the first average eye-opening value AV1 and the real-time average eye-opening value AVR.

According to an embodiment of the present disclosure, since the processor 120 averages the real-time average eye-opening value and the previous average eye-opening value to generate an updated average eye-opening value, the phenomenon of excessive changes in the average eye-opening value due to misjudgment can be suppressed.

Returning to FIG. 5, the processor 120 averages the eye-closing values within a predetermined period to generate an average eye-closing value (step S504). In addition, the processor 120 further updates the eye-opening threshold value to the weighted average of the average eye-opening value and the average eye-closing value (step S505).

As shown in FIG. 6, since the average eye-closing value AVCL is not generated at the second point of time T2, the second eye-opening threshold value TH2 is the second average eye-opening value AV2 multiplied by a ratio. In addition, the processor 120 averages the first eye-opening value OP1 and the second eye-opening value OP2 within the predetermined time TD that are less than the eye-opening threshold value TH to generate a first average eye-closing value AVCL1. In other words, before the first average eye-closing value AVCL1 is generated, the average eye-closing value AVCL is zero, and the processor 120 uses the average of the eye-opening values OP within each predetermined time TD less than the eye-opening threshold value TH to update the average eye-closing value AVCL. According to some embodiments of the present disclosure, the predetermined time is approximately 10 to 15 seconds.

Next, after the first average eye-closing value AVCL1 is generated, the processor 120 uses the weighted average of the first average eye-closing value AVCL1 and the corresponding average eye-opening value AVOP to generate a new eye-opening threshold value TH. According to some embodiments of the present disclosure, the eye-open threshold value TH is the sum of the eyes-open average value AVOP multiplied by the first weighting factor W1 and the average eye-closing value multiplied by the second weighting factor W2, where the sum of the first weighting factor W1 and the second weighting factor W2 is 1. The eye-opening threshold value TH is shown in Eq. 2:

$$TH = AVOP \times W 1 + AVCL 1 \times W 2 \qquad ( Eq . 2 )$$

For example, when the processor 120 operates in the low sensitivity mode, the first weighting factor W1 is smaller than the second weighting factor W2. When the processor 120 operates in the medium sensitivity mode, the first weighting factor W1 may be equal to the second weighting factor W2. When the processor 120 operates in the high sensitivity mode, the first weighting factor W1 is greater than the second weighting factor W2.

In other words, when operating in the high-sensitivity mode, the eye-opening threshold value TH is closer to the average eye-opening value AVOP, making it easier for the processor 120 to determine that the user 10 is in an eye-closing state. When operating in the low-sensitivity mode, the eye-opening threshold TH is closer to the average eye-closing value AVCL1, making it less easy for the processor 120 to determine that the user 10 is in an eye-closing state, thereby avoiding misjudgment.

Returning to FIG. 5, after step S505, the processor 120 determines whether the updated average eye-opening value is less than the initial eye-opening threshold value (step S506). When the updated average eye-opening value is less than the initial average eye-opening value, the processor 120 adjusts the average eye-opening value to the average of the average eye-opening value and the initial average eye-opening value (step S507). When the updated average eye-opening value is not less than the initial eye-opening threshold value, the processor 120 further determines whether the eye-opening threshold value exceeds the initial average eye-opening value (step S508).

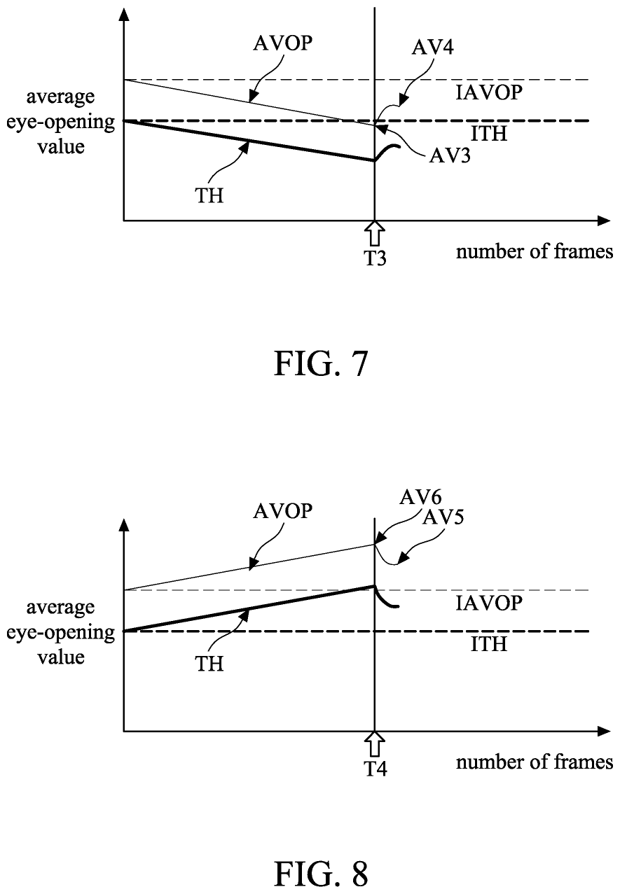

When the eye-opening threshold value exceeds the initial average eye-opening value, the processor 120 adjusts the average eye-opening value to the average of the average eye-opening value and the initial average eye-opening value (step S507). When the eye-opening threshold value does not exceed the initial average eye-opening value, the processor 120 does not adjust the average eye-opening value and returns to step S503 to update the average eye-opening value. After step S507, the processor 120 also returns to step S503 to update the average eye-opening value. The detailed operation from step S506 to step S508 will be described in detail with reference to FIG. 7 in the following paragraphs.

FIG. 7 is a schematic diagram showing the average eye-opening value and the eye-opening threshold value in accordance with an embodiment of the present disclosure. As shown in FIG. 7, the processor 120 generates the initial average eye-opening value IAVOP in step S501 and generates the initial eye-opening threshold value ITH in step S502. In addition, as the number of frames increases, the average eye-opening value AVOP and the eye-opening threshold value TH gradually decrease from the initial average eye-opening value IAVOP and the initial eye-opening threshold value ITH respectively, and the average eye-opening value AVOP is smaller than the initial eye-opening threshold value ITH at the third time T3.

When the processor 102 determines in step S506 that the average eye-opening value AVOP is less than the initial eye-opening threshold value ITH at the third time T3, the processor 120 updates the average eye-opening value AVOP to the fourth average eye-opening value AV4, where the fourth average eye-opening value AV4 is the average of the third average eye-opening value AV3 at the third time T3 and the initial average eye-opening value IAVOP, as shown in Eq. 3. According to some embodiments of the present disclosure, when the processor 120 adjusts the eye-open average value AVOP, the eye-open threshold value TH will also be adjusted accordingly since the eye-open threshold value TH is the weighted average of the eye-open average value AVOP and the average eye-closing value AVCL.

$$AV 4 = \frac{AV 3 + I AVOP}{2} \qquad ( Eq . 3 )$$

FIG. 8 is a schematic diagram showing the average eye-opening value and the eye-opening threshold value in accordance with another embodiment of the present disclosure. As shown in FIG. 8, as the number of frames increases, the average eye-opening value AVOP and the eye-opening threshold value TH gradually increase from the initial average eye-opening value IAVOP and the initial eye-opening threshold value ITH respectively, and the eye-opening threshold value TH exceeds the initial eye-opening average IA VOP at the fourth time T4.

When the processor 120 determines in step S508 that the eye-opening threshold value TH exceeds the initial average eye-opening value IAVOP at the fourth time T4, the processor 120 updates the average eye-opening value AVOP to the fifth average eye-opening value AV5, where the fifth average eye-opening value AV5 is the average of the sixth average eye-opening value AV6 at the fourth time T4 and the initial average eye-opening value IAVOP, as shown in Eq. 4. According to some embodiments of the present disclosure, when the processor 120 adjusts the eye-open average value AVOP, the eye-opening threshold value TH is also adjusted accordingly since the eye-open threshold value TH is the weighted average of the average eye-opening value AVOP and the average eye-closing value AVCL.

$$AV 5 = \frac{AV 6 + I AVOP}{2} \qquad ( Eq . 4 )$$

According to some embodiments of the present disclosure, as shown in FIGS. 7 and 8, since the average eye-opening value AVOP may become too large or too small over time, it leads that the eye-opening threshold value TH becomes an unreasonable value. By using the initial average eye-opening value IAVOP and the initial eye-opening threshold value ITH as the upper and lower limits and maintaining the average eye-opening value AVOP and the eye-opening threshold value TH to vary within a range, it helps to maintain the rationality of the average eye-opening value AVOP and the threshold value TH, so as to improve the accuracy of the human-eye state detection method.

According to an embodiment of the present disclosure, the memory 130 in FIG. 1 is further configured to store the eye-opening threshold values TH, the average eye-opening values AVOP, the eye closing average values AVCL, the initial average eye-opening IA VOP, and initial eye-opening threshold value ITH corresponding to the left eye area 221 and the right eye area 222.

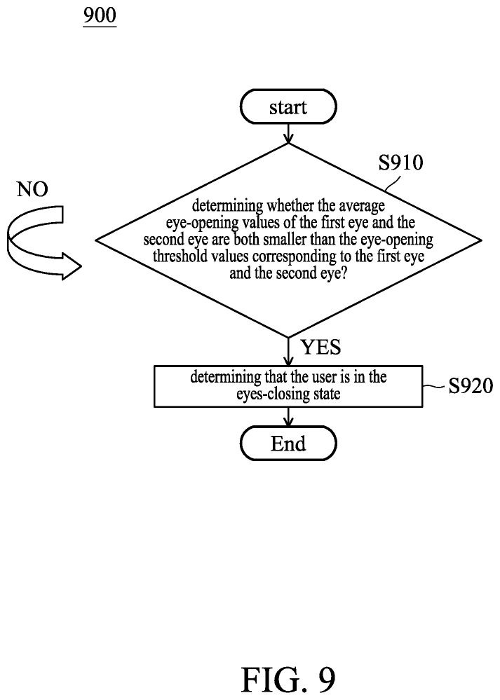

FIG. 9 is a flowchart showing a determination procedure in accordance with′ an embodiment of the present disclosure. As shown in the determination procedure 900 in FIG. 9, the processor 120 determines whether the average eye-opening values of the left eye area 221 and the right eye area 222 are both smaller than the corresponding eye-opening threshold value (step S910). When the processor 120 determines that the average eye-opening values of the left eye area 221 and the right eye area 222 is both smaller than the corresponding eye-opening threshold values, the processor 120 determines that the user 10 is in the eye-closing state (step S920).

In other words, when the processor 120 determines that both eyes of the user 10 are in the eye-closing state, it will be determined that the user 10 is in the eye-closing state. When the processor 120 determines that the average eye-opening value of either the left eye area 221 or the right eye area 222 is not less than the corresponding eye-opening threshold value, the processor 120 repeatedly executes step S910.

According to some embodiments of the present disclosure, when the processor 120 determines that the user 10 is in the eye-closing state in step S920, the processor 120 may issue a warning to notify the user 10. According to some embodiments of the present disclosure, when the processor 120 executes step S910, the processor 120 simultaneously executes the update procedure 500 to update the average eye-opening value AVOP, the average eye-closing value AVCL, and the eye-opening threshold value TH in real time, and determines whether the user 10 is in the eye-closing state based on the average eye-opening value AVOP and the eye-opening threshold value TH right at the moment of step S910.

According to other embodiments of the present disclosure, when the processor 120 does not detect the face area 210 for more than a predetermined period and then detects the face area 210 once again, the processor 120 re-executes the human-eye state detection method 400, and recalculates the average eye-opening value AVOP and the eye-opening threshold value TH. According to other embodiments of the present disclosure, when the human-eye state detection method 400 is suspended and then restarted, the average eye-opening value AVOP and the eye-opening threshold value TH are going to be recalculated.

According to other embodiments of the present disclosure, when executing step S420 to step S460, step S501 to step S508, and step S910 to step S920, the processor 120 may repeatedly determine whether the execution condition has been met. When it is determined that execution condition has not been met, the processor 120 can interrupt the human-eye state detection method 400, the update procedure 500, and the determination procedure 900 at any time, clear the average eye-opening value AVOP, the initial average eye-opening value IAVOP, and the initial eye-opening threshold value ITH, and eye-opening threshold value TH of the memory 130, and return to step S410 to repeatedly determine whether the execution condition has been met again to re-execute the human-eye state detection method 400.

A human-eye state detection device and a human-eye state detection method thereof are proposed herein. By continuously updating the eye-opening threshold values of the left eye and the right eye, there is a representative eye-opening threshold value as the basis for determination in the process of detecting the state of the human eyes. In addition, by dynamically adjusting the eye-opening thresholds of the left and right eyes, it helps to significantly reduce the impact of the external environment. Regardless of whether the user's left and right eyes are the same size, whether the eyes are disturbed by the shadow of other things (such as hair), whether the user wears glasses, whether the lens has an elevation or depression angle, and other individual factors and environmental factors, it can accurately determine the state of human eyes. Furthermore, the possibility of misjudgment would be significantly reduced since the state of eyes of the user is determined based on the states of the left eye and the right eye, thereby improving the user experience.

Although some embodiments of the present disclosure and their advantages have been described in detail, it should be understood that various changes, substitutions and alterations can be made herein without departing from the spirit and scope of the disclosure as defined by the appended claims. For example, it will be readily understood by those skilled in the art that many of the features, functions, processes, and materials described herein may be varied while remaining within the scope of the present disclosure. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A human-eye state detection device, comprising:
an image capturing device, continuously capturing a plurality of frames of facial images of a user; and
a processor, determining an eye area from the plurality of frames of facial images and calculating an average eye-opening value based on the eye area;
wherein the processor repeatedly updates an eye-opening threshold value based on the average eye-opening value;
wherein in response to the average eye-opening value being less than the eye-opening threshold value, the processor determines that the user is in an eye-closing state.

2. The human-eye state detection device as defined in claim 1, wherein in response to the processor determining that an execution condition has been met, the processor calculates an eye-opening value from the eye area of each frame and generates the average eye-opening value by averaging the eye-opening values of a specific amount of frames at a first point of time;
wherein the average eye-opening value generated at the first point of time is a first average eye-opening value;
wherein the processor multiplies the first average eye-opening value by a ratio to generate an initial eye-opening threshold value and takes the initial eye-opening threshold value as the eye-opening threshold value.

3. The human-eye state detection device as defined in claim 2, wherein after the processor takes the initial eye-opening threshold value as the eye-opening threshold value, the processor uses an average of the eye-opening values of the specific amount of frames at a second point of time to generate a real-time average eye-opening value, thereby updating the average eye-opening value to a second average eye-opening value;
wherein the eye-opening threshold value is the second average eye-opening value multiplied by the ratio;
wherein the second average eye-opening value is an average of the first average eye-opening value and the real-time average eye-opening value.

4. The human-eye state detection device as defined in claim 2, wherein the processor further averages a plurality of eye-closing values within a predetermined period to generate an average eye-closing value;
wherein in the predetermined period, the eye-closing value is the eye-opening value being less than the eye-opening threshold value;
wherein the processor updates the eye-opening threshold value to a weighted average of the average eye-opening value and the average eye-closing value.

5. The human-eye state detection device as defined in claim 4, wherein the eye-opening threshold value is a sum of the average eye-opening value multiplied by a first weighted factor and the average eye-closing value multiplied by a second weighted factor;
wherein a sum of the first weighted factor and the second weighted factor is 1;
wherein in response to the processor operating in a first sensitivity mode, the first weighted factor is a first value;
wherein in response to the processor operating in a second sensitivity mode, the first weighted factor is a second value;
wherein the first value and the second value are different;
wherein in response to the processor operating in the first sensitivity mode, the ratio is a first ratio value;
wherein in response to the processor operating in the second sensitivity mode, the ratio is a second ratio value;
wherein the first ratio value and the second ratio value are different.

6. The human-eye state detection device as defined in claim 3, wherein the processor further determines whether the second average eye-opening value is less than the initial eye-opening threshold value;
wherein in response to the second average eye-opening value being less than the initial eye-opening threshold value, the processor adjusts the average eye-opening value to the average of the first average eye-opening value and the second average eye-opening value.

7. The human-eye state detection device as defined in claim 6, wherein in response to the second average eye-opening value not being less than the initial eye-opening value, the processor further determines whether the eye-opening threshold value exceeds the first average eye-opening value;
wherein in response to the eye-opening threshold exceeding the first average eye-opening value, the processor adjusts the average eye-opening value to the average of the first average eye-opening value and the second average eye-opening value;
wherein in response to the eye-opening threshold not exceeding the first average eye-opening value, the processor repeatedly updates the average eye-opening value in real-time;
wherein the eye-opening threshold value varies with the average eye-opening value.

8. The human-eye state detection device as defined in claim 2, further comprising:
a detection device, detecting a moving speed; and
a memory, storing the average eye-opening value, the first average eye-opening value, the initial eye-opening value, and the eye-opening threshold value;
wherein in response to the moving speed exceeding a predetermined speed, the processor determines that the execution condition has been met;
wherein in response to the execution condition not being met, the processor erases the average eye-opening value, the first average eye-opening value, the initial eye-opening threshold value, and the eye-opening threshold value in the memory.

9. The human-eye state detection device as defined in claim 8, wherein the processor further determines, by morphology, whether the eye area is in an eye-opening state;
wherein in response to the eye area is in the eye-opening state and the moving speed exceeding the predetermined speed, the processor determines that the execution condition has been met.

10. The human-eye state detection device as defined in claim 1, wherein the processor further determines a left eye area and a right eye area from the eye area and generates the average eye-opening values and the eye-opening threshold values corresponding to the left eye area and the right eye area based on the left eye area and the right eye area respectively;
wherein in response to the processor determining that the average eye-opening value of the left eye area and the average eye-opening value of the right eye area are both less than the corresponding eye-opening threshold values, the processor determines that the user is in the eye-closing state.

11. A human-eye state detection method adapted to a human-eye state detection device, wherein the human-eye state detection device comprises an image capturing device, wherein the human-eye state detection method comprises:
continuously capturing a plurality of frames of facial images of a user using the image capturing device;
determining an eye area from the plurality of frames of the facial images and calculating an average eye-opening value based on the eye area;
based on the average eye-opening value, updating an eye-opening threshold value in real time; and
in response to the average eye-opening value being less than the eye-opening threshold value, determining that the user is in an eye-closing state.

12. The human-eye state detection method as defined in claim 11, further comprising:
determining whether an execution condition has been met;
in response to determining that the execution condition has been met, calculating an eye-opening value from the eye area of each frame and averaging the eye-opening values of a specific amount of frames at a first point of time to generate the average eye-opening value;
wherein the average eye-opening value generated at the first point of time is a first average eye-opening value; and
multiplying the first average eye-opening value by a ratio to generate an initial eye-opening threshold value as the eye-opening threshold value.

13. The human-eye state detection method as defined in claim 12, further comprising:
after the step of multiplying the first average eye-opening value by the ratio to generate the initial eye-opening threshold value as the eye-opening threshold value, generating a real-time average eye-opening value by averaging the eye-opening values of the specific amount of frames at a second point of time to update the average eye-opening value to a second average eye-opening value;
wherein the eye-opening threshold value is the second average eye-opening value multiplied by the ratio;
wherein the second average eye-opening value is an average of the first average eye-opening value and the real-time average eye-opening value.

14. The human-eye state detection method as defined in claim 12, further comprising:
averaging a plurality of eye-closing values within a predetermined period in order to generate an average eye-closing value;
wherein the eye-opening value within the predetermined period being less than the eye-opening threshold value is the eye-closing value; and
updating the eye-opening threshold value to a weighted average of the average eye-opening value and the average eye-closing value.

15. The human-eye state detection method as defined in claim 14, wherein the eye-opening threshold value is a sum of the average eye-opening value multiplied by a first weighted factor and the average eye-closing value multiplied by a second weighted factor;
wherein a sum of the first weighted factor and the second weighted factor is 1;
wherein in response to the human-eye state detection device operating in a first sensitivity, the first weighted factor is a first value;
wherein in response to the human-eye state detection device operating in a second sensitivity, the second weighted factor is a second value;
wherein the first value and the second value are different;
wherein in response to the processor operating in the first sensitivity, the ratio is a first ratio value;
wherein in response to the processor operating in the second sensitivity, the ratio is a second ratio value;
wherein the first ratio value and the second ratio value are different.

16. The human-eye state detection method as defined in claim 13, further comprising:
determining whether the second average eye-opening value is less than the initial eye-opening threshold value; and
in response to the second average eye-opening value being less than the initial eye-opening threshold value, adjusting the average eye-opening value to an average of the first average eye-opening value and the second average eye-opening value.

17. The human-eye state detection method as defined in claim 16, further comprising:
in response to the second average eye-opening value not being less than the initial eye-opening threshold value, further determining whether the eye-opening threshold value exceeds the first average eye-opening value;
in response to the eye-opening threshold value exceeding the first average eye-opening value, adjusting the average eye-opening value to an average of the first average eye-opening value and the second average eye-opening value; and
in response to the eye-opening threshold value not exceeding the first average eye-opening value, repeatedly updating the average eye-opening value;
wherein the eye-opening threshold value varies with the average eye-opening value.

18. The human-eye state detection method as defined in claim 12, further comprising:
detecting a moving speed using a detection device;
storing the average eye-opening value, the first average eye-opening value, the initial eye-opening threshold value, and the eye-opening threshold value by using a memory;
in response to the moving speed exceeding a predetermined speed, determining that the execution condition has been met; and
in response to the execution condition not being satisfied, erasing the average eye-opening value, the first average eye-opening value, the initial eye-opening threshold value, and the eye-opening threshold value that are stored.

19. The human-eye state detection method as defined in claim 18, further comprising:
determining whether the eye area is in an eye-opening state by morphology; and
wherein in response to the eye area being in the eye-opening state and the moving speed exceeding the predetermined speed, determining that the execution condition has been met.

20. The human-eye state detection method as defined in claim 11, further comprising:
determining a left eye area and a right eye area from the eye area;
generating the average eye-opening values and the eye-opening threshold values corresponding to the left eye area and the right eye area based on the left eye area and the right eye area; and
in response to determining that the average eye-opening value corresponding to the left eye area and the average eye-opening value corresponding to the right eye area both are less than the corresponding eye-opening threshold values, determining that the user is in the eye-closing state.
